(19) United States
(12) Patent Application Publication
(10) Pub. No.: US 2022/0358750 A1
(43) Pub. Date: Nov. 10, 2022

(54) LEARNING DEVICE, DEPTH INFORMATION ACQUISITION DEVICE, ENDOSCOPE SYSTEM, LEARNING METHOD, AND PROGRAM

(71) Applicant: FUJIFILM Corporation, Tokyo (JP)

(72) Inventor: Takayuki TSUJIMOTO, Kanagawa (JP)

(73) Assignee: FUJIFILM Corporation, Tokyo (JP)

(21) Appl. No.: 17/730,783

(22) Filed: Apr. 27, 2022

(30) Foreign Application Priority Data
May 6, 2021 (JP) 2021-078694

(51) Int. Cl. G06V 10/774; G06T 7/55; G06T 17/00; G06T 5/00; G06T 7/00; G06V 10/22

(52) U.S. Cl. CPC G06V 10/774; G06T 7/55; G06T 17/00; G06T 5/001; G06T 7/0012; G06V 10/22; G06T 2207/10068; G06T 2207/20081; G06T 2207/30004; G06V 2201/03

(57) ABSTRACT

Provided are a learning device, a depth information acquisition device, an endoscope system, a learning method, and a program capable of efficiently acquiring a learning data set used for machine learning to perform depth estimation, and capable of implementing a highly accurate depth estimation for an actually imaged endoscope image.

The learning device includes a processor performing endoscope image acquisition processing of acquiring an endoscope image obtained by imaging a body cavity with an endoscope system, actual measurement information acquisition processing of acquiring actually measured first depth information corresponding to at least one measurement point in the endoscope image, imitation image acquisition processing of acquiring an imitation image obtained by imitating an image of the body cavity to be imaged with the endoscope system, imitation depth acquisition processing of acquiring second depth information including depth information of one or more regions in the imitation image, and learning processing of causing a learning model to perform learning by using a first learning data set and a second learning data set.

### CROSS-REFERENCE TO RELATED APPLICATION

The present application claims priority under 35 U.S.C § 119(a) to Japanese Patent Application No. 2021-078694 filed on May 6, 2021, which is hereby expressly incorporated by reference, in its entirety, into the present application.

### BACKGROUND OF THE INVENTION

### 1. Field of the Invention

The present invention relates to a learning device, a depth information acquisition device, an endoscope system, a learning method, and a program.

### 2. Description of the Related Art

In recent years, it has been attempted to assist a doctor's diagnosis by using artificial intelligence (AI) in a diagnosis using an endoscope system. For example, AI is used to perform an automatic lesion detection for the purpose of reducing oversight of lesions by doctors, and AI is also used to perform an automatic identification of lesions and the like for the purpose of reducing the number of biopsies.

In such use of AI, AI is made to perform recognition processing on a motion picture (frame image) observed by a doctor in real time to assist diagnosis.

On the other hand, an endoscope image captured by an endoscope system is often imaged by a monocular camera attached to a distal end of an endoscope. Therefore, it is difficult for doctors to obtain depth information from endoscope images, which makes diagnosis or surgery using the endoscope system difficult. Therefore, a technique for estimating depth information from endoscope images of a monocular camera using AI has been proposed (WO2020/189334A).

### SUMMARY OF THE INVENTION

In order to make AI (a recognizer configured with a trained model) estimate depth information, it is necessary to prepare a learning data set in which an endoscope image and the depth information corresponding to the endoscope image are defined as a set as correct answer data. Thereafter, it is necessary to prepare a large number of learning data sets and make AI to perform machine learning.

However, since it is not easy to actually measure and acquire the accurate depth information of the entire image, it is difficult to prepare a large number of learning data sets and train AI.

On the other hand, an image imitating an endoscope image and the corresponding depth information thereof can be generated relatively easily by simulation or the like. Therefore, it is conceivable that the learning is performed by using the learning data set generated by the simulation or the like instead of the actually measured learning data set. However, in a case where the learning is performed only with the learning data set generated by the simulation or the like, it is not possible to guarantee the estimation performance of the depth information in a case where the endoscope image obtained by actually imaging an examination target is input.

The embodiment of the present invention has been made in view of such circumstances, and an object thereof is to provide a learning device, a depth information acquisition device, an endoscope system, a learning method, and a program capable of efficiently acquiring a learning data set used for machine learning to perform depth estimation, and capable of implementing highly accurate depth estimation for an actually imaged endoscope image.

A learning device according to an aspect of the present invention comprises a processor, and a learning model that estimates depth information of an endoscope image, in which the processor is configured to perform endoscope image acquisition processing of acquiring the endoscope image obtained by imaging a body cavity with an endoscope system, actual measurement information acquisition processing of acquiring actually measured first depth information corresponding to at least one measurement point in the endoscope image, imitation image acquisition processing of acquiring an imitation image obtained by imitating an image of the body cavity to be imaged with the endoscope system, imitation depth acquisition processing of acquiring second depth information including depth information of one or more regions in the imitation image, and learning processing of causing the learning model to perform learning by using a first learning data set composed of the endoscope image and the first depth information, and a second learning data set composed of the imitation image and the second depth information.

According to the present aspect, the learning model performs the learning by using the first learning data set composed of the endoscope image and the first depth information, and the second learning data set composed of the imitation image and the second depth information. As a result, it is possible to efficiently acquire the learning data set used for the learning model to perform the learning, and it is possible to implement highly accurate depth estimation for the actually imaged endoscope image.

Preferably, the first depth information is acquired by using an optical range finder provided at a distal end of an endoscope of the endoscope system.

Preferably, the imitation image and the second depth information are acquired based on pseudo three-dimensional computer graphics of the body cavity.

Preferably, the imitation image is acquired by imaging a model of the body cavity with the endoscope system, and the second depth information is acquired based on three-dimensional information of the model.

Preferably, the processor is configured to make a first loss weight during the learning processing using the first learning data set and a second loss weight during the learning processing using the second learning data set different from each other.

Preferably, the first loss weight is larger than the second loss weight.

A depth information acquisition device according to another aspect of the present invention comprises a trained model in which learning is performed in the learning device described above.

According to the present aspect, an actually imaged endoscope image is input, and highly accurate depth estimation can be output.

An endoscope system according to still another aspect of the present invention comprises the depth information acquisition device described above, an endoscope, and a processor, in which the processor is configured to perform image acquisition processing of acquiring an endoscope image captured with the endoscope, image input processing of inputting the endoscope image to the depth information acquisition device, and estimation processing of causing the depth information acquisition device to estimate depth information of the endoscope image.

According to the present aspect, an actually imaged endoscope image is input, and highly accurate depth estimation can be output.

Preferably, the endoscope system further comprises a correction table corresponding to a second endoscope that differs at least in objective lens from a first endoscope with which the endoscope image of the first learning data set is acquired, in which the processor is configured to perform correction processing of correcting the depth information, which is acquired in the estimation processing, by using the correction table in a case where an endoscope image is acquired with the second endoscope.

According to the present aspect, even in a case where an endoscope image obtained by imaging with the endoscope, which is different from the endoscope acquired the learning data (endoscope image) obtained in a case where the learning is performed on the depth information acquisition device, is input, it is possible to acquire highly accurate depth information.

A learning method according to still another aspect of the present invention is a learning method using a learning device that includes a processor and a learning model that estimates depth information of an endoscope image, the learning method comprises the following steps executed by the processor, an endoscope image acquisition step of acquiring the endoscope image obtained by imaging a body cavity with an endoscope system, an actual measurement information acquisition step of acquiring actually measured first depth information corresponding to at least one measurement point in the endoscope image, an imitation image acquisition step of acquiring an imitation image obtained by imitating an image of the body cavity to be imaged with the endoscope system, an imitation depth acquisition step of acquiring second depth information including depth information of one or more regions in the imitation image, and a learning step of causing the learning model to perform learning by using a first learning data set composed of the endoscope image and the first depth information, and a second learning data set composed of the imitation image and the second depth information.

A program according to still another aspect of the present invention is a program for causing a learning device that includes a processor and a learning model that estimates depth information of an endoscope image to execute a learning method, the program causing the processor to execute an endoscope image acquisition step of acquiring the endoscope image obtained by imaging a body cavity with an endoscope system, an actual measurement information acquisition step of acquiring actually measured first depth information corresponding to at least one measurement point in the endoscope image, an imitation image acquisition step of acquiring an imitation image obtained by imitating an image of the body cavity to be imaged with the endoscope system, an imitation depth acquisition step of acquiring second depth information including depth information of one or more regions in the imitation image, and a learning step of causing the learning model to perform learning by using a first learning data set composed of the endoscope image and the first depth information, and a second learning data set composed of the imitation image and the second depth information.

According to the embodiment of the present invention, the learning model performs the learning by using the first learning data set composed of the endoscope image and the first depth information, and the second learning data set composed of the imitation image and the second depth information. As a result, it is possible to efficiently acquire the learning data set used for the learning model to perform the learning, and it is possible to implement highly accurate depth estimation for the actually imaged endoscope image.

### BRIEF DESCRIPTION OF THE DRAWINGS

FIG. 1 is a block diagram showing an example of a configuration of a learning device of the present embodiment.

FIG. 2 is a block diagram showing a main function implemented by a processor in the learning device.

FIG. 3 is a flow chart showing each step of a learning method.

FIG. 4 is a schematic diagram showing an example of the overall configuration of an endoscope system capable of acquiring a first learning data set.

FIG. 5 is a view describing an example of an endoscope image and first depth information.

FIG. 6 is a view describing acquisition of depth information of a measurement point L in an optical range finder.

FIGS. 7A and 7B are views showing an example of an imitation image.

FIGS. 8A and 8B are views describing second depth information corresponding to the imitation image.

FIG. 9 is a view conceptually showing a model of a human large intestine.

FIG. 10 is a functional block diagram showing main functions of a learning model and a learning unit.

FIG. 11 is a view describing processing of the learning unit in a case where learning is performed by using the first learning data set.

FIG. 12 is a functional block diagram showing the main functions of the learning unit and the learning model of the present example.

FIG. 13 is a block diagram showing an embodiment of an image processing device equipped with a depth information acquisition device.

FIG. 14 is a diagram showing a specific example of a correction table.

### DESCRIPTION OF THE PREFERRED EMBODIMENTS

Hereinafter, preferred embodiments of a learning device, a depth information acquisition device, an endoscope system, a learning method, and a program according to the embodiments of the present invention will be described with reference to the accompanying drawings.

### First Embodiment

A first embodiment of the present invention is a description of a learning device.

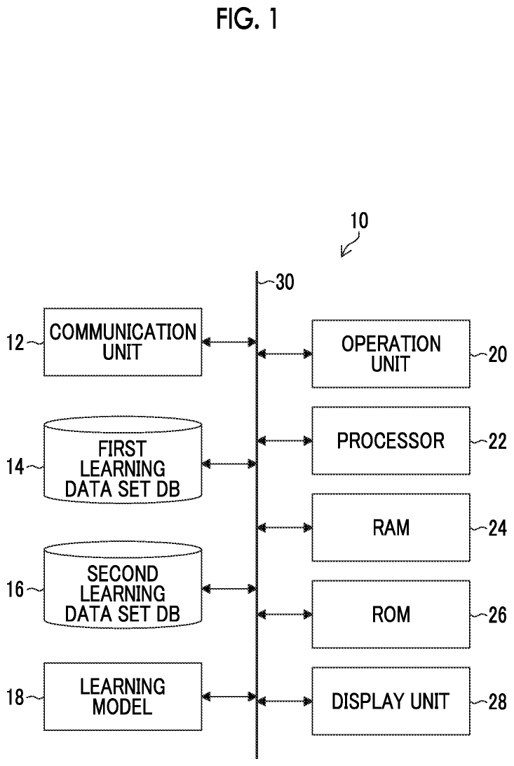

FIG. 1 is a block diagram showing an example of a configuration of the learning device of the present embodiment.

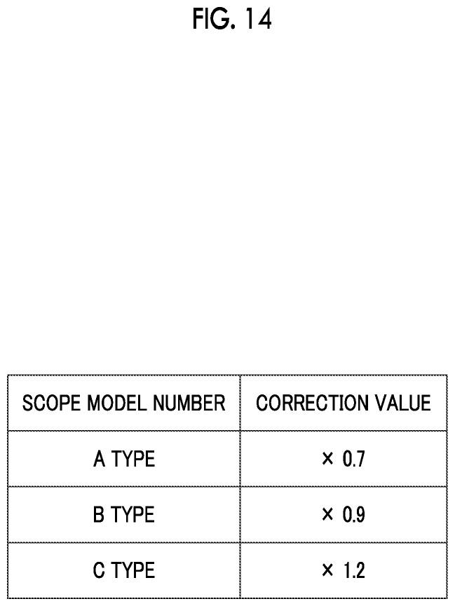

The learning device 10 is composed of a personal computer or a workstation. The learning device 10 is composed of a communication unit 12, a first learning data set database (described as a first learning data set DB in the FIG. 14, a second learning data set database (described as a second learning data set DB in the FIG. 16, a learning model 18, an operation unit 20, a processor 22, a random access memory (RAM) 24, a read only memory (ROM) 26, and a display unit 28. Each unit is connected via a bus 30. In the present example, an example in which each unit is connected to the bus 30 has been described, but the example of the learning device 10 is not limited to this. For example, a part or all of the learning device 10 may be connected via a network. Here, the network includes various communication networks such as a local area network (LAN), a wide area network (WAN), and the Internet.

The communication unit 12 is an interface for performing communication processing with an external device by wire or wirelessly and exchanging information with the external device.

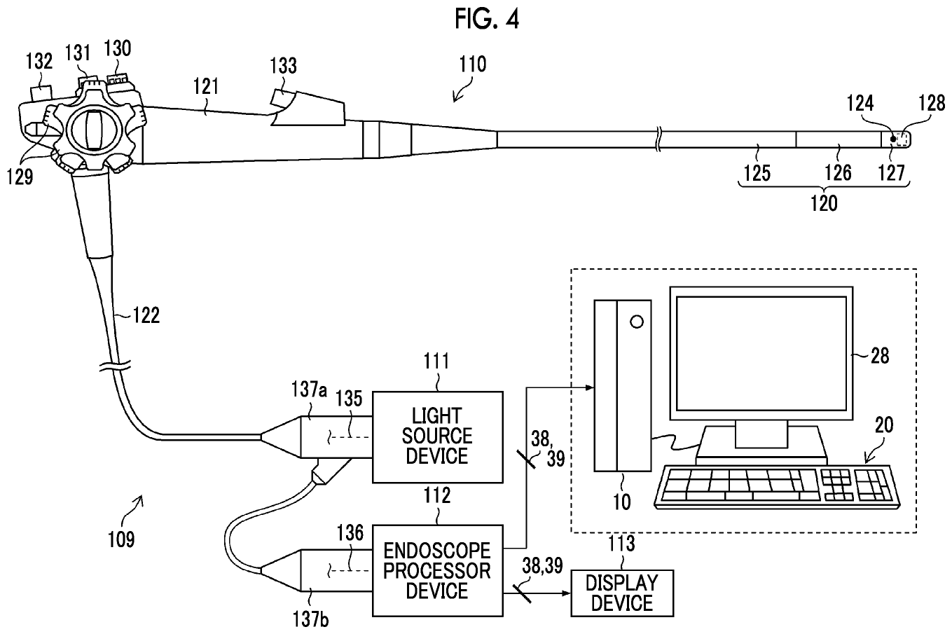

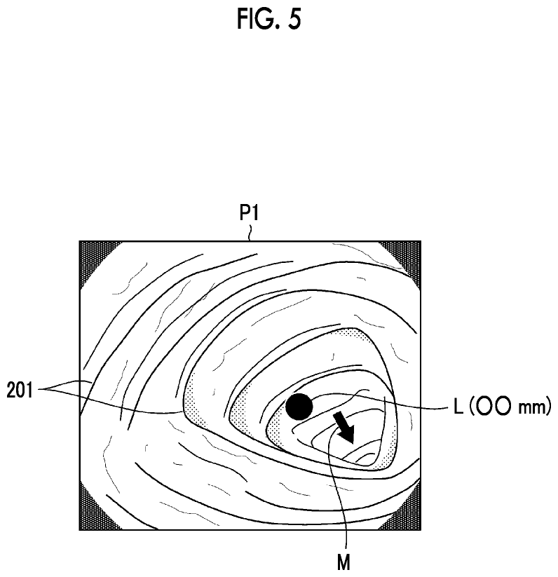

The first learning data set database 14 stores the endoscope image and corresponding first depth information. Here, the endoscope image is an image obtained by imaging a body cavity that is actually an examination target with an endoscope 110 (see FIG. 4) of the endoscope system 109. Further, the first depth information is actually measured depth information corresponding to at least one measurement point of the endoscope image. The first depth information is acquired, for example, by an optical range finder 124 of the endoscope 110. The endoscope image and the first depth information constitute a first learning data set. The first learning data set database 14 stores a plurality of first learning data sets.

The second learning data set database 16 stores an imitation image and corresponding second depth information. Here, the imitation image is an image obtained by imitating the endoscope image captured the body cavity that is the examination target, with the endoscope system 109. Further, the second depth information is depth information of one or more regions of the imitation image. The second depth information is preferably depth information of one or more regions wider than the measurement point of the first depth information. For example, it is preferable that the entire region having the second depth information occupies 50% or more of the imitation image or 80% or more of the imitation image. Furthermore, it is more preferable that the entire region having the second depth information is the entire image of the imitation image. In the following description, a case where the entire image of the imitation image has the second depth information will be described. The imitation image and the second depth information constitute a second learning data set. The second learning data set database 16 stores a plurality of second learning data sets. The first learning data set and the second learning data set will be described in detail later.

The learning model 18 is composed of one or a plurality of convolutional neural networks (CNNs). In the learning model 18, the endoscope image is input, and machine learning is performed so as to output the depth information of the entire image of the received endoscope image. Here, the depth information is information related to a distance between a subject, which is captured in the endoscope image, and a camera (imaging element 128 (FIG. 4)). The learning model 18 mounted on the learning device 10 is untrained, and the learning device 10 performs the machine learning for causing the learning model 18 to perform an estimation of the depth information of the endoscope image. As the structure of the learning model 18, various known models are used, for example, U-Net is used.

The operation unit 20 is an input interface that receives various operation inputs with respect to the learning device 10. As the operation unit 20, a keyboard, a mouse, or the like that is connected to a computer by wire or wireless, is used.

The processor 22 is composed of one or a plurality of central processing units (CPUs). The processor 22 reads various programs stored in the ROM 26 or a hard disk apparatus (not shown) and executes various processing. The RAM 24 is used as a work area for the processor 22. Further, the RAM 24 is used as a storage unit for temporarily storing the read programs and various data. The learning device 10 may configure the processor 22 with a graphics processing unit (GPU).

The ROM 26 permanently stores a computer boot program, a program such as a basic input/output system (BIOS), data, or the like. Further, the RAM 24 temporarily stores programs, data, or the like loaded from the ROM 26, a storage device connected separately, or the like, and includes a work area used by the processor 22 to perform various processing.

The display unit 28 is an output interface on which necessary information for the learning device 10 is displayed. As the display unit 28, various monitors such as a liquid crystal monitor that can be connected to a computer are used.

Here, an example in which the learning device 10 is composed of a single personal computer or a workstation has been described, but the learning device 10 may be composed of a plurality of personal computers.

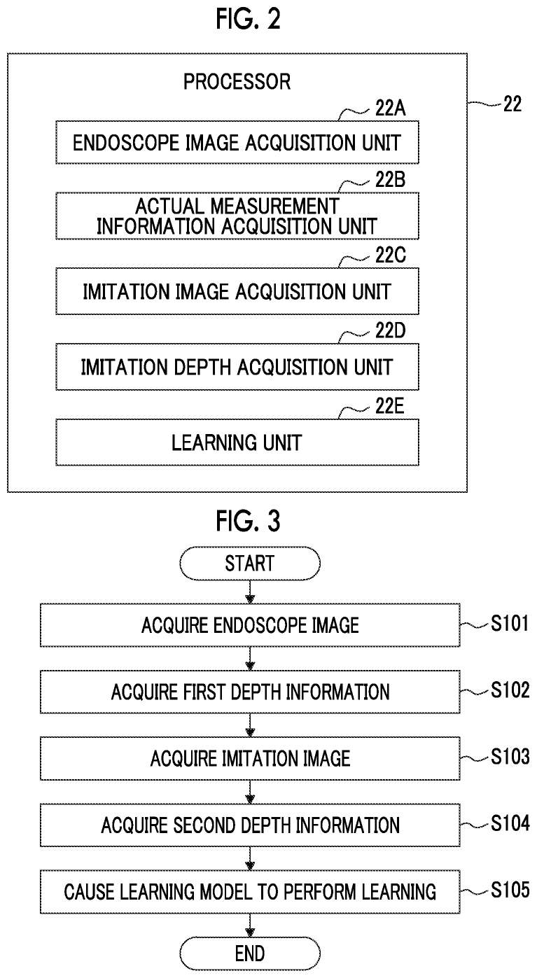

FIG. 2 is a block diagram showing a main function implemented by the processor 22 in the learning device 10.

The processor 22 is mainly composed of an endoscope image acquisition unit 22A, an actual measurement information acquisition unit 22B, an imitation image acquisition unit 22C, an imitation depth acquisition unit 22D, and a learning unit 22E.

The endoscope image acquisition unit 22A performs endoscope image acquisition processing. The endoscope image acquisition unit 22A acquires the endoscope image stored in the first learning data set database 14.

The actual measurement information acquisition unit 22B performs actual measurement information acquisition processing. The actual measurement information acquisition unit 22B acquires the actually measured first depth information corresponding to at least one measurement point of the endoscope image stored in the first learning data set database 14.

The imitation image acquisition unit 22C performs imitation image acquisition processing. The imitation image acquisition unit 22C acquires the imitation image stored in the second learning data set database 16.

The imitation depth acquisition unit 22D performs imitation depth acquisition processing. The imitation depth acquisition unit 22D acquires the second depth information stored in the second learning data set database 16.

The learning unit 22E performs learning processing on the learning model 18. The learning unit 22E causes the learning model 18 to perform learning by using the first learning data set and the second learning data set. Specifically, the learning unit 22E optimizes a parameter of the learning model 18 based on a loss in a case where the learning is performed by the first learning data set and a loss in a case where the learning is performed by the second learning data set.

Next, a learning method using the learning device 10 (each step of the learning method is performed by executing a program by the processor 22 of the learning device 10) will be described.

FIG. 3 is a flow chart showing each step of the learning method.

First, the endoscope image acquisition unit 22A acquires the endoscope image from the first learning data set database 14 (step S101: endoscope image acquisition step). Next, the actual measurement information acquisition unit 22B acquires the first depth information from the first learning data set database 14 (step S102: actual measurement information acquisition step). Thereafter, the imitation image acquisition unit 22C acquires the imitation image from the second learning data set database 16 (step S103: imitation image acquisition step). Further, the imitation depth acquisition unit 22D acquires the second depth information from the second learning data set database 16 (step S104: imitation depth acquisition step). Thereafter, the learning unit 22E causes the learning model 18 to perform the learning by using the first learning data set and the second learning data set (step S105: learning step).

Next, the first learning data set and the second learning data set will be described in detail.

First Learning Data Set

The first learning data set is composed of the endoscope image and the first depth information.

FIG. 4 is a schematic diagram showing an example of the overall configuration of the endoscope system capable of acquiring the first learning data set (the endoscope image and the first depth information).

As shown in FIG. 4, the endoscope system 109 includes an endoscope 110 that is an electronic endoscope, a light source device 111, an endoscope processor device 112, and a display device 113. Further, in the learning device 10, the endoscope system 109 is connected, and the endoscope images (a motion picture 38 and a static image 39) imaged with the endoscope 110 are transmitted.

The endoscope 110 images time-series endoscope images including a subject image, and is, for example, an endoscope for a lower or upper gastrointestinal tract. The endoscope 110 includes an insertion part 120 that is inserted into a subject (for example, the large intestine) and has a distal end and a proximal end, a hand operation unit 121 that is installed consecutively to the proximal end side of the insertion part 120 and is gripped by a doctor who is an operator to perform various operations, and a universal cord 122 that is installed consecutively to the hand operation unit 121.

The entire insertion part 120 has a small diameter and is formed in a long shape. The insertion part 120 is configured in which a flexible soft portion 125, a bendable part 126 capable of bending by operating the hand operation unit 121, and a tip part 127, which is provided with an imaging optical system (objective lens) (not shown), an imaging element 128, and an optical range finder 124, are installed consecutively in order from the proximal end side to the distal end side of the insertion part 120.

The imaging element 128 is a complementary metal oxide semiconductor (CMOS) type or charge coupled device (CCD) type imaging element Image light of a site to be observed is incident on an imaging surface of the imaging element 128 through an observation window (not shown) opened on a distal end surface of the tip part 127, and an objective lens (not shown) disposed behind the observation window. The imaging element 128 images the image light (converted into an electric signal) of the site to be observed incident on the imaging surface of the imaging element 128, and outputs an imaging signal. That is, the endoscope images are sequentially imaged by the imaging element 128.

The optical range finder 124 acquires the first depth information. Specifically, the optical range finder 124 optically measures the depth of the subject captured in the endoscope image. For example, the optical range finder 124 is composed of a light amplification by stimulated emission of radiation (LASER) range finder or a light detection and ranging (LiDAR) range finder. The optical range finder 124 acquires the actually measured first depth information corresponding to the measurement point of the endoscope image acquired by the imaging element 128. It is preferable that the number of measurement points is at least one, and more preferably two or three points. Further, the measurement points are preferably 10 points or less. Further, the imaging of the endoscope image with the imaging element 128 and the acquisition of the depth information of the optical range finder 124 may be performed at the same time, or the acquisition of the depth information may be performed before and after the imaging of the endoscope image.

The hand operation unit 121 is provided with various operation members operated by a doctor (user). Specifically, the hand operation unit 121 is provided with two types of bending operation knobs 129 used for bending operation of the bendable part 126, an air/water supply button 130 for air/water supply operation, and a suction button 131 for suction operation. Further, the hand operation unit 121 is provided with a static image-imaging instruction unit 132 for performing an imaging instruction of a static image 39 of a site to be observed, and a treatment tool inlet port 133 for inserting a treatment tool (not shown) into a treatment tool insertion path (not shown) that is inserted through the insertion part 120.

The universal cord 122 is a connection cord for connecting the endoscope 110 to the light source device 111. The universal cord 122 includes a light guide 135, a signal cable 136, and a fluid tube (not shown) that are inserted through the insertion part 120. Further, at an end of the universal cord 122, a connector 137a, which is connected to the light source device 111, and a connector 137b, which is branched from the connector 137a and connected to the endoscope processor device 112, are provided.

By connecting the connector 137a to the light source device 111, the light guide 135 and the fluid tube (not shown) are inserted into the light source device 111. In this way, necessary illumination light, water, and gas are supplied from the light source device 111 to the endoscope 110 via the light guide 135 and the fluid tube (not shown). As a result, the site to be observed is irradiated with the illumination light from the illumination window (not shown) on the distal end surface of the tip part 127. Further, in response to the above-mentioned pressing operation of the air/water supply button 130, gas or water is injected from the air and water supply nozzle (not shown) on the distal end surface of the tip part 127 toward the observation window (not shown) on the distal end surface.

By connecting the connector 137b to the endoscope processor device 112, the signal cable 136 and the endoscope processor device 112 are electrically connected to each other. As a result, the imaging signal of the site to be observed is output from the imaging element 128 of the endoscope 110 to the endoscope processor device 112 via the signal cable 136, and a control signal is output from the endoscope processor device 112 to the endoscope 110.

The light source device 111 supplies the illumination light to the light guide 135 of the endoscope 110 via the connector 137a. As the illumination light, light in various wavelength ranges is selected according to the purpose of observation, for example, white light (light in the white wavelength range or light in a plurality of wavelength ranges), light in one or a plurality of specific wavelength ranges, or a combination thereof.

The endoscope processor device 112 controls the operation of the endoscope 110 via the connector 137b and the signal cable 136. Further, the endoscope processor device 112 generates the motion picture 38 consisting of a time-series frame image 38a including a subject image based on the imaging signal acquired from the imaging element 128 of the endoscope 110 via the connector 137b and the signal cable 136. Further, in a case where the static image-imaging instruction unit 132 is operated by the hand operation unit 121 of the endoscope 110, the endoscope processor device 112 generates the static image 39 according to a timing of the imaging instruction from one frame image 38a in the motion pictures 38 in parallel with the generation of the motion picture 38.

In the present description, the motion picture (frame image 38a) 38 and the static image 39 are defined as the endoscope images obtained by imaging the inside of the subject, that is, the body cavity. Further, in a case where the motion picture 38 and the static image 39 are images obtained by the above-mentioned light in the specific wavelength range (special light), both the motion picture 38 and the static image 39 are special light images. The endoscope processor device 112 outputs the generated motion picture 38 and the static image 39 to the display device 113 and the learning device 10.

The endoscope processor device 112 may generate a special light image having information related to the specific wavelength range described above based on a normal light image obtained by the white light described above. In this case, the endoscope processor device 112 functions as a special light image acquisition unit. The endoscope processor device 112 obtains a signal of the specific wavelength range by performing an operation based on color information of red, green, and blue [red, green, blue (RGB)] or cyan, magenta, and yellow [cyan, magenta, yellow (CMY)] included in the normal light image.

Further, the endoscope processor device 112 may generate a feature amount image such as a known oxygen saturation image based on at least one of the above-mentioned normal light image obtained by white light or the above-mentioned special light image obtained by light in the specific wavelength range (special light), for example. In this case, the endoscope processor device 112 functions as a feature amount image generation unit. The motion picture 38 or the static image 39 including an in-vivo image, the normal light image, the special light image, and the feature amount image is an endoscope image obtained by imaging a human body for the purpose of diagnosis and examination, or by imaging the measured results.

The display device 113 is connected to the endoscope processor device 112 and functions as the display unit for displaying the motion picture 38 and the static image 39 input from the endoscope processor device 112. The doctor performs an advance or retreat operation or the like of the insertion part 120 while checking the motion picture 38 displayed on the display device 113 and operates the static image-imaging instruction unit 132 to perform imaging of the static image of the site to be observed, and perform treatments such as diagnosis and biopsy in a case where a lesion is found in a site to be observed.

FIG. 5 is a view describing an example of the endoscope image and the first depth information.

The endoscope image P1 is an image captured with the above-mentioned endoscope system 109. Specifically, the endoscope image P1 is an image obtained by imaging a part of the human large intestine, which is an examination target, with the imaging element 128 attached to the tip part 127 of the endoscope 110. The endoscope image P1 shows the folds 201 of the large intestine and shows a part of the large intestine that continues in a tubular shape in the direction of the arrow M. Further, FIG. 5 shows the first depth information D1 (“OO mm”) corresponding to the measurement point L of the endoscope image P1. The first depth information D1 is the depth information corresponding to the measurement point L on the endoscope image P1 in this way. A position of the measurement point L may be set in advance such as in the center of the image or may be appropriately set by the user.

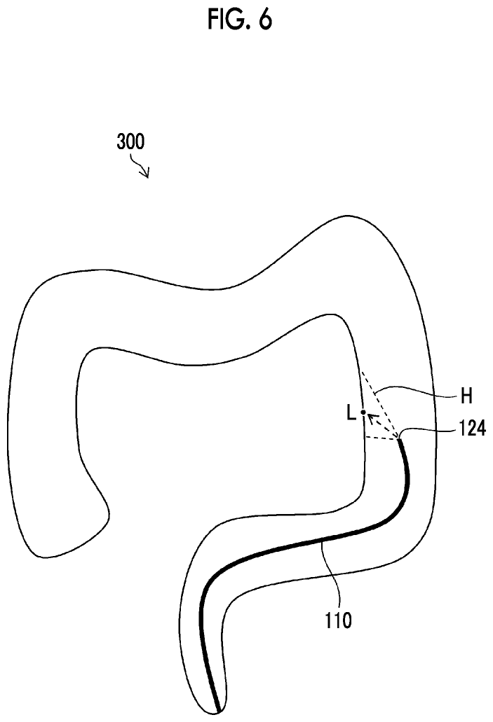

FIG. 6 is a view describing the acquisition of the depth information of the measurement point L in the optical range finder 124.

FIG. 6 shows a mode in which the endoscope 110 is inserted into the large intestine 300 and the endoscope image P1 is imaged. The endoscope 110 acquires the endoscope image P1 by imaging the large intestine 300 within a range of an angle of view H. Further, a distance (depth information) to the measurement point L is acquired by the optical range finder 124 provided at the tip part 127 of the endoscope 110.

As described above, the endoscope system 109 including the optical range finder 124 acquires the endoscope image P1 and the first depth information D1 constituting the first learning data set. Since the first learning data set is composed of the endoscope image P1 and the depth information of the measurement point L in this way, the first learning data set can be easily acquired as compared with a case where the depth information of the entire image of the endoscope image P1 is acquired. In the above description, an example in which the first learning data set is acquired with the endoscope system 109 has been described, but the embodiment is not limited to this example. The first learning data set may be acquired by another method as long as the actually measured first depth information corresponding to the endoscope image and at least one measurement point on the endoscope image can be acquired.

Second Learning Data Set

The second learning data set is composed of the imitation image and the second depth information. In the following description, an example in which the imitation image and the depth information of the entire image of the imitation image (second depth information) are acquired based on a three-dimensional computer graphics will be described.

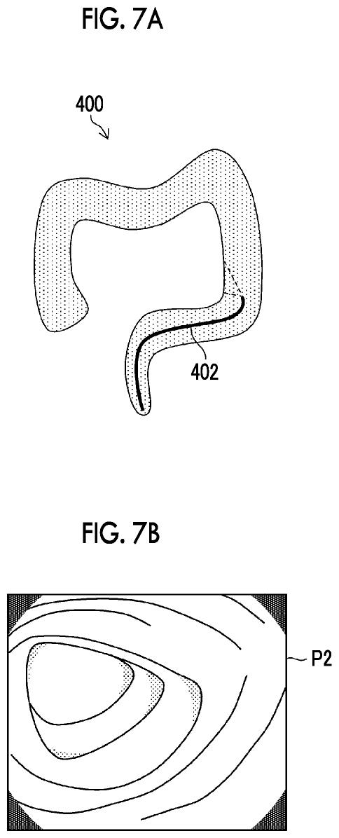

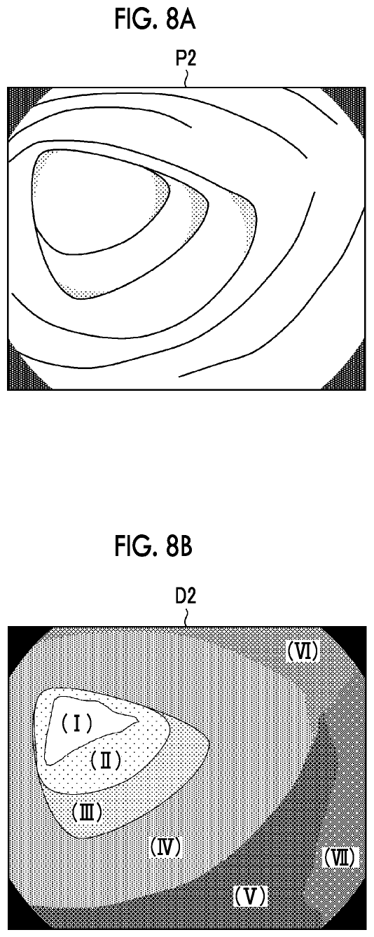

FIGS. 7A and 7B are views showing an example of the imitation image. FIG. 7A shows pseudo three-dimensional computer graphics 400 imitating the human large intestine, and FIG. 7B shows an imitation image P2 obtained based on the three-dimensional computer graphics 400.

The three-dimensional computer graphics 400 is generated by imitating the human large intestine using the computer graphics technique. Specifically, the three-dimensional computer graphics 400 has a general (representative) color, shape, and size (three-dimensional information) of the human large intestine. Therefore, it is possible to generate the imitation image P2 by simulating the fact that the human large intestine is imaged by the virtual endoscope 402 based on the three-dimensional computer graphics 400. The imitation image P2 shows a color scheme and a shape such that the human large intestine is imaged with the endoscope system 109 based on the three-dimensional computer graphics 400. Further, as described below, by specifying a position of the virtual endoscope 402 based on the three-dimensional computer graphics 400, the depth information (second depth information) of the entire image of the imitation image P2 can be generated. The three-dimensional computer graphics 400 can be generated by using data acquired by a plurality of imaging apparatuses different from each other. For example, the three-dimensional computer graphics 400 may determine the shape and size of the large intestine from a three-dimensional shape model of the large intestine generated from an image acquired by a computed tomography (CT) or a magnetic resonance imaging (MRI), or may determine the color of the large intestine from an image that is imaged with the endoscope.

FIGS. 8A and 8B are views describing the second depth information corresponding to the imitation image P2. FIG. 8A shows the imitation image P2 described with reference to FIG. 7B, and FIG. 8B shows the second depth information D2 corresponding to the imitation image P2.

Since the three-dimensional computer graphics 400 has three-dimensional information, the depth information of the entire image of the imitation image P2 (second depth information D2) can be acquired by specifying the position of the virtual endoscope 402.

The second depth information D2 is the depth information of the entire image corresponding to the imitation image P2. The second depth information D2 is divided into each region (I) to (VII) according to the depth information, and each region has different depth information. The second depth information D2 only needs to have the depth information related to the entire image of the corresponding imitation image P2 and is not limited to being divided into the regions (I) to (VII). For example, the second depth information D2 may have the depth information for each pixel or may have the depth information for each of a plurality of pixels.

As described above, the imitation image P2 and the second depth information D2 constituting the second learning data set are generated based on the three-dimensional computer graphics 400. Therefore, the second depth information D2 is generated relatively easily as compared with the case of acquiring the depth information of the entire image of the actual endoscope image.

In the above-mentioned example, the case where the imitation image P2 and the second depth information are generated based on the three-dimensional computer graphics 400 has been described, but the generation of the imitation image P2 and the second depth information is not limited to this example. Hereinafter, another example of the generation of the second learning data set will be described.

For example, instead of the three-dimensional computer graphics 400, a model (phantom) imitating the human large intestine may be created, and the imitation image P2 may be acquired by imaging the model with the endoscope system 109.

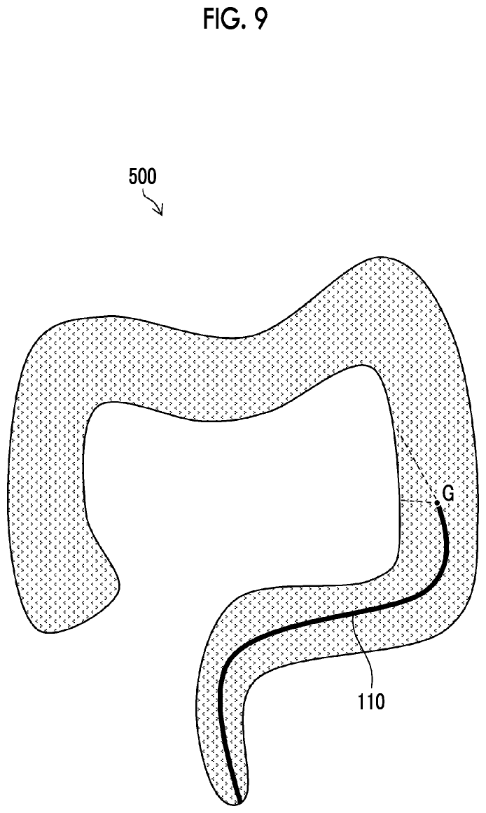

FIG. 9 is a view conceptually showing a model of a human large intestine.

The model 500 is a model created by imitating the human large intestine. Specifically, the inside of the model 500 has a color, shape, and the like similar to the human large intestine. Therefore, the imitation image P2 can be acquired by inserting the endoscope 110 of the endoscope system 109 into the model 500 and imaging the model 500. Further, the model 500 has general (representative) three-dimensional information of the human large intestine. Therefore, by acquiring a position G (x1, y1, z1) of the imaging element 128 of the endoscope 110, the depth information (second depth information) of the entire image of the imitation image P2 can be obtained using the three-dimensional information of the model 500.

As described above, the imitation image P2 and the second depth information D2 constituting the second learning data set are acquired based on the model 500. Therefore, the second depth information is generated relatively easily as compared with the case of acquiring the depth information of the entire image of the actual endoscope image.

Learning Step

Next, the learning step (step S105) performed by the learning unit 22E will be described. In the learning step, learning is performed on the learning model 18 using the first learning data set and the second learning data set.

First Example of Learning Step

First, a first example of the learning step will be described. In the present example, the endoscope image P1 and the imitation image P2 are input to the learning model 18, and learning (machine learning) is performed on the learning model 18.

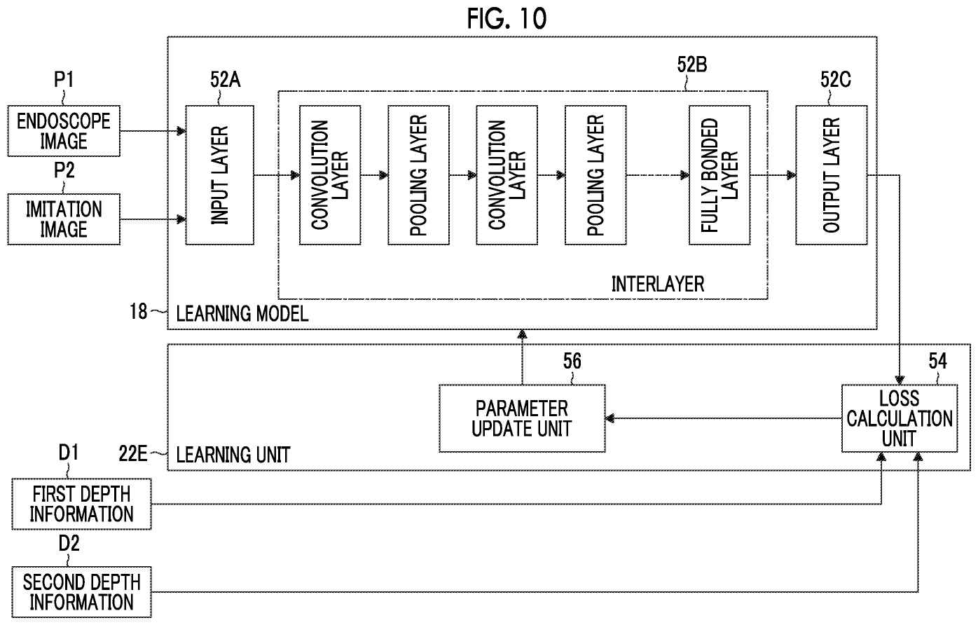

FIG. 10 is a functional block diagram showing main functions of the learning model 18 and the learning unit 22E. The learning unit 22E includes a loss calculation unit 54 and a parameter update unit 56. Further, the first depth information D1 is input to the learning unit 22E as correct answer data for learning performed by inputting the endoscope image P1. Further, the second depth information D2 is input to the learning unit 22E as correct answer data for learning performed by inputting the imitation image P2.

As the learning progresses, the learning model 18 becomes a depth information acquisition device that outputs the depth information of the entire image from the endoscope image. The learning model 18 has a plurality of layer structures and stores a plurality of weight parameters. The learning model 18 is changed from an untrained model to a trained model by updating the weight parameter from an initial value to an optimum value.

The learning model 18 includes an input layer 52A, an interlayer 52B, and an output layer 52C. The input layer 52A, the interlayer 52B, and the output layer 52C each have a structure in which a plurality of “nodes” are connected by “edges”. The endoscope image P1 and the imitation image P2, which are learning targets, are input to the input layer 52A, respectively.

The interlayer 52B is a layer for extracting features from an image input from the input layer 52A. The interlayer 52B has a plurality of sets, in which a convolution layer and a pooling layer are defined as one set, and a fully bonded layer. The convolution layer performs a convolution operation, in which a filter is used with respect to a node near the previous layer, and acquires a feature map. The pooling layer reduces the feature map output from the convolution layer to make a new feature map. The fully bonded layer bonds all the nodes of the immediately preceding layer (here, the pooling layer). The convolution layer plays a role in feature extraction such as edge extraction from an image, and the pooling layer plays a role in imparting robustness such that the extracted features are not affected by parallel translation or the like. The interlayer 52B is not limited to the case where the convolution layer and the pooling layer are defined as one set but includes a case where the convolution layers are continuous and a normalization layer.

The output layer 52C is a layer that outputs the depth information of the entire image of the endoscope image based on the features extracted by the interlayer 52B.

The trained learning model 18 outputs the depth information of the entire image of the endoscope image.

Any initial values are set for a filter coefficient and an offset value, which are applied to each convolution layer of the before-trained learning model 18, and a weight of the connection between the fully bonded layer and the next layer thereof.

The loss calculation unit 54 acquires the depth information output from the output layer 52C of the learning model 18 and the correct answer data (first depth information D1 or second depth information D2) with respect to the input image, and calculates a loss between the depth information and the correct answer data. As a method for calculating the loss, for example, the soft max cross entropy, the least squared error (mean squared error (MSE)), or the like can be considered.

The parameter update unit 56 adjusts the weight parameter of the learning model 18 by using the loss back propagation method based on the loss calculated by the loss calculation unit 54. The parameter update unit 56 can set a first loss weight during the learning processing using the first learning data set and a second loss weight during the learning processing using the second learning data set. For example, the parameter update unit 56 may make the first loss weight and the second loss weight the same or may make the first loss weight and the second loss weight different from each other. In a case where the first loss weight and the second loss weight are made different, the parameter update unit 56 makes the first loss weight larger than the second loss weight. As a result, the learning results obtained by using the actually imaged endoscope image P1 can be more reflected.

This parameter adjustment processing is repeated, and learning is repeated until the difference between the depth information output by the learning model 18 and the correct answer data (first depth information and second depth information) becomes small.

Here, the learning is performed on the learning model 18 so as to output the depth information of the entire image of the input endoscope image. On the other hand, the first depth information D1, which is the correct answer data of the first learning data set, has only the depth information of the measurement point L. Therefore, in the case where the learning is performed with the first learning data set, the loss calculation unit 54 does not use anything other than the depth information at the measurement point L for learning (set as don't care processing).

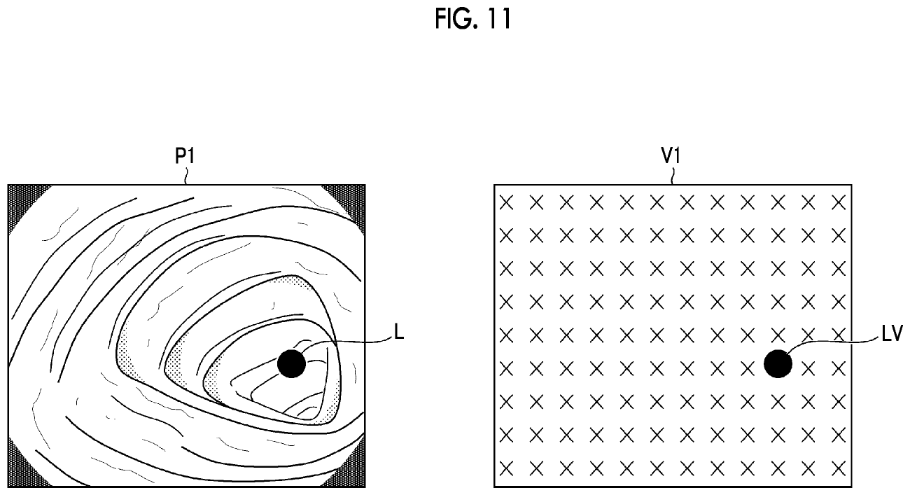

FIG. 11 is a view describing processing of the learning unit 22E in a case where learning is performed by using the first learning data set.

In a case where the endoscope image P1 is input, the learning model 18 outputs the estimated depth information V1. The estimated depth information V1 is the depth information in the entire image of the endoscope image P1. Here, the first depth information, which is the correct answer data of the endoscope image P1, has only the depth information of a portion corresponding to the measurement point L. Therefore, in a case where learning is performed using the first learning data set, the loss calculation unit 54 does not use depth information other than the depth information LV at the portion corresponding to the measurement point L for learning. That is, the depth information other than the depth information LV at the portion corresponding to the measurement point L does not affect the calculation of the loss by the loss calculation unit 54. In this way, by performing learning using only the depth information LV at the portion corresponding to the measurement point L for learning, the learning of the learning model 18 can be efficiently performed even in a case where there is no depth information (correct answer data) for the entire image.

The learning unit 22E uses the first learning data set and the second learning data set to optimize each parameter of the learning model 18. In the learning of the learning unit 22E, a certain number of first learning data sets and second learning data sets may be extracted, batch processing of learning may be performed by the extracted first learning data set and the second learning data set, and a mini-batch method, in which the extracting and the batch processing are repeated, may be used.

As described above, in the present example, the endoscope image P1 and the imitation image P2 are each input to one learning model 18, and the machine learning is performed.

Second Example of Learning Step

Next, a second example of the learning step will be described. In the present example, a learning model 18 that performs a multitask by branching into a task to perform classification and a task to perform segmentation in the latter stage of the learning model 18, is used.

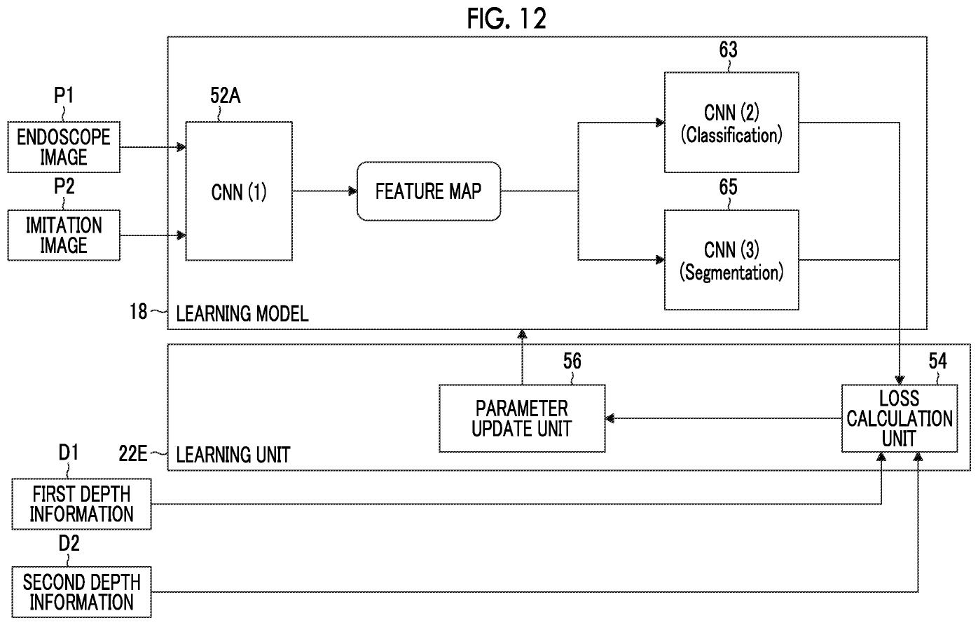

FIG. 12 is a functional block diagram showing the main functions of the learning unit 22E and the learning model 18 of the present example. The portions already described in FIG. 10 are designated by the same reference numerals and the description thereof will be omitted.

The learning model 18 is composed of a CNN(1) 61, a CNN(2) 65, and a CNN(3) 67. Each of the CNN(1) 61, CNN(2) 65, and CNN(3) 67 is configured with a convolutional neural network (CNN).

The endoscope image P1 and the imitation image P2 are input to the CNN(1) 61. The CNN(1) 61 outputs a feature map for each of the input endoscope image P1 and imitation image P2.

In a case where the endoscope image P1 is input to the CNN(1) 61, the feature map is input to the CNN(2) 63. The CNN(2) 63 is a model for performing learning of the classification. The CNN(2) 63 inputs the output result to the loss calculation unit 54. The loss calculation unit 54 calculates a loss between the output result of the CNN(2) 63 and the first depth information D1. Thereafter, the parameter update unit 56 updates parameters of the learning model 18 based on the calculation result from the loss calculation unit 54.

On the other hand, in a case where the imitation image P2 is input to the CNN(1) 61, the feature map is input to the CNN(3) 65. The CNN(3) 65 is a model for performing learning of the segmentation. Further, the CNN(3) 65 inputs the output result to the loss calculation unit 54. The loss calculation unit 54 calculates a loss between the output result of the CNN(3) 65 and the second depth information D2. Thereafter, the parameter update unit 56 updates parameters of the learning model 18 based on the calculation result from the loss calculation unit 54.

As described above, in learning, the learning that uses the endoscope image P1 and the learning that uses the imitation image P2 are respectively performed in different tasks by using the learning model 18 in which the task is branched into the classification and the segmentation in the latter stage. As a result, efficient learning can be performed by using the first learning data set and the second learning data set.

### Second Embodiment

Next, a second embodiment of the present invention will be described. The present embodiment is regarding a depth information acquisition device composed of the learning model 18 (trained model) in which learning is performed in the learning device 10. According to the depth information acquisition device of the present embodiment, it is possible to provide the user with highly accurate depth information.

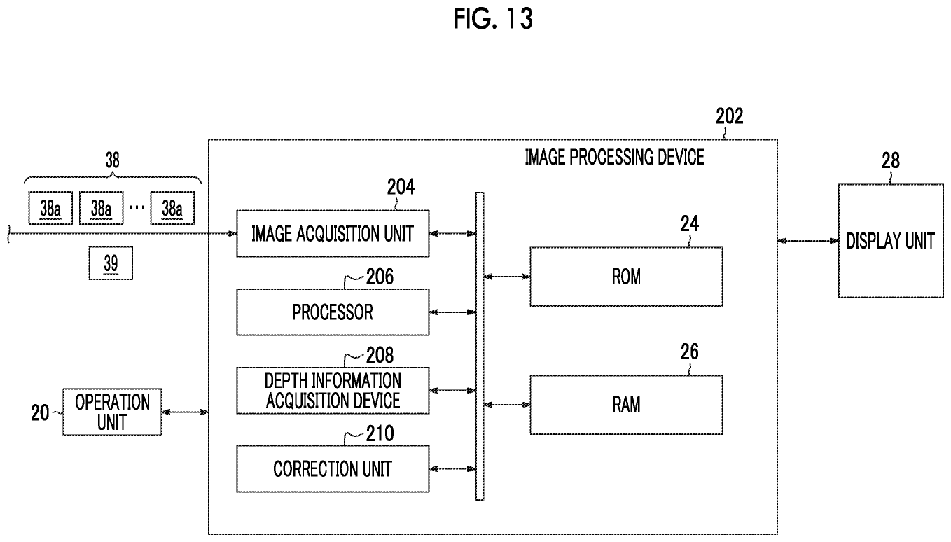

FIG. 13 is a block diagram showing the embodiment of an image processing device equipped with the depth information acquisition device. The portions already described in FIG. 1 are designated by the same reference numerals and the description thereof will be omitted.

The image processing device 202 is mounted on the endoscope system 109 described with reference to FIG. 4. Specifically, the image processing device 202 is connected in place of the learning device 10 connected to the endoscope system 109. Therefore, the motion picture 38 and the static image 39 imaged with the endoscope system 109 are input to the image processing device 202.

The image processing device 202 is composed of an image acquisition unit 204, a processor 206, a depth information acquisition device 208, a correction unit 210, a RAM 24, and a ROM 26.

The image acquisition unit 204 acquires the endoscope image captured with the endoscope 110 (image acquisition processing). Specifically, the image acquisition unit 204 acquires the motion picture 38 or the static image 39 as described above.

The processor (central processing unit) 206 performs each processing of the image processing device 202. For example, the processor 206 causes the image acquisition unit 204 to acquire the endoscope image (motion picture 38 or static image 39) (image acquisition processing). Further, the processor 206 inputs the acquired endoscope image to the depth information acquisition device 208 (image input processing). Further, the processor 206 causes the depth information acquisition device 208 to estimate the depth information of the received endoscope image (estimation processing). The processor 206 is composed of one or a plurality of CPUs.

As described above, the depth information acquisition device 208 is composed of a trained model in which the learning is performed on the learning model 18 with the first learning data set and the second learning data set. In the depth information acquisition device 208, the endoscope image (motion picture 38, static image 39) acquired by the endoscope 110 is input, and the input depth information of the endoscope image is output. The depth information acquired by the depth information acquisition device 208 is the input depth information of the entire image of the endoscope.

The correction unit 210 corrects the depth information estimated with the depth information acquisition device 208 (correction processing). In a case where the endoscope image, which is acquired with the endoscope (second endoscope) different from the endoscope (first endoscope) 109 where the endoscope image used during the learning of the learning model 18 is acquired, is input to the depth information acquisition device 208, it is possible to acquire more accurate depth information by correcting the depth information. Since the endoscope image is different even in a case where the same subject is imaged due to the difference in the endoscope, it is preferable to correct the depth information output according to the endoscope. Here, the difference in the endoscope means that at least the objective lens is different, and as described above, this is a case where different endoscope images are acquired even in a case where the same subject is imaged.

The correction unit 210 corrects the depth information output from the depth information acquisition device 208 by using, for example, the correction table stored in advance. The correction table will be described later.

The display unit 28 displays the endoscope images (motion picture 38 and static image 39) acquired by the image acquisition unit 204. Further, the display unit 28 displays the depth information acquired by the depth information acquisition device 208 or the depth information corrected by the correction unit 210. In this way, the user can recognize the depth information corresponding to the displayed endoscope image by displaying the depth information or the corrected depth information on the display unit 28.

FIG. 14 is a diagram showing a specific example of the correction table. The correction table can be obtained by inputting the endoscope images obtained by the respective endoscopes into the depth information acquisition device 208 in advance and acquiring and comparing the depth information.

In the correction table, a correction value is changed according to a model number of the endoscope. Specifically, in a case where the endoscope image is acquired by using an A-type endoscope and the depth information is estimated based on the endoscope image, the corrected depth information is acquired by applying the correction value (×0.7) to the estimated depth information. Further, in a case where the endoscope image is acquired by using a B-type endoscope and the depth information is estimated based on the endoscope image, the corrected depth information is acquired by applying the correction value (×0.9) to the estimated depth information. Further, in a case where the endoscope image is acquired by using a C-type endoscope and the depth information is estimated based on the endoscope image, the corrected depth information is acquired by applying the correction value (×1.2) to the estimated depth information. In this way, by correcting the depth information with the correction table having a correction value according to the endoscope, it is possible to acquire highly accurate depth information even with endoscope images acquired with various endoscopes.

As described above, since the depth information acquisition device 208 of the present embodiment is composed of the learning model 18 (trained model) in which the learning is performed in the learning device 10, it is possible to provide the user with highly accurate depth information.

Others

Others 1

In the above description, the embodiment in which the image processing device 202 includes the correction unit 210 has been described. However, in a case where the endoscope, in which the endoscope image input to the learning model 18 during the learning is imaged, and the endoscope, in which the endoscope image input to the depth information acquisition device 208 is imaged, are the same, the correction unit 210 may not be included in the image processing device 202. Further, in a case where the accuracy of the estimated depth information is within an allowable range even in a case where the endoscope, in which the endoscope image input to the learning model 18 during the learning is imaged, and the endoscope, in which the endoscope image input to the depth information acquisition device 208 is imaged, are different, the correction unit 210 may not be included in the image processing device 202.

Others 2

In the above description, the case where the depth information estimated by the depth information acquisition device 208 is corrected by the correction unit 210 has been described. However, in a case where the endoscope, in which the endoscope image input to the learning model 18 during the learning is imaged, and the endoscope, in which the endoscope image input to the depth information acquisition device 208 is imaged, are different, the correction may be performed by another method. For example, the endoscope image input to the depth information acquisition device 208 may be converted into an endoscope image input to the learning model 18. For example, conversion is performed in advance by using an image conversion technique such as pix2pix. Thereafter, the depth information acquisition device 208 may perform an estimation of the depth information by inputting the converted endoscope image. As a result, even in a case where the endoscope, in which the endoscope image used during the learning is imaged, and the endoscope, in which the endoscope image used during performing depth estimation after learning is imaged, are different, it is possible to perform an estimation of accurate depth information.

Others 3

In the above description, the case where only the endoscope image is input to the depth information acquisition device 208 to estimate the depth information has been described. However, other information may be input to the depth information acquisition device 208 to estimate the depth information of the endoscope image. For example, in a case where the optical range finder 124 is provided like the endoscope 110 described above, the depth information acquired by the optical range finder 124 may be also input to the depth information acquisition device 208 together with the endoscope image. In this case, the learning model 18 performs learning for estimating the depth information with the endoscope image and the depth information of the optical range finder 124.

Others 4

In the above embodiment, the hardware-like structure of the processing unit (for example, the endoscope image acquisition unit 22A, the actual measurement information acquisition unit 22B, the imitation image acquisition unit 22C, the imitation depth acquisition unit 22D, the learning unit 22E, the image acquisition unit 204, the depth information acquisition device 208, the correction unit 210) that executes various processing is various processors as shown below. Various processors include a central processing unit (CPU), which is a general-purpose processor that executes software (programs) and functions as various processing units, a programmable logic device (PLD), which is a processor whose circuit configuration is able to be changed after manufacturing such as a field programmable gate array (FPGA), a dedicated electric circuit, which is a processor having a circuit configuration specially designed to execute specific processing such as an application specific integrated circuit (ASIC), and the like.

One processing unit may be composed of one of these various processors or may be composed of two or more processors of the same type or different types (for example, a plurality of FPGAs or a combination of a CPU and an FPGA). Further, a plurality of processing units may be composed of one processor. As an example of configuring a plurality of processing units with one processor, first, as represented by a computer such as a client or a server, there is a form in which one processor is configured by a combination of one or more CPUs and software, and this processor functions as a plurality of processing units. Second, as represented by a system on chip (SoC) or the like, there is a form in which a processor, which implements the functions of the entire system including a plurality of processing units with one integrated circuit (IC) chip, is used. In this way, the various processing units are configured by using one or more of the above-mentioned various processors as a hardware-like structure.

Further, the hardware-like structure of these various processors is, more specifically, an electric circuit (circuitry) in which circuit elements such as semiconductor elements are combined.

Each of the above configurations and functions can be appropriately implemented by any hardware, software, or a combination of both. For example, the embodiment of the present invention can be applied to a program that causes a computer to execute the above processing steps (processing procedures), a computer-readable recording medium (non-transitory recording medium) on which such a program is recorded, or a computer on which such a program can be installed.

Although the example of the present invention has been described above, it is needless to say that the embodiment of the present invention is not limited to the above-described embodiments and various modifications can be made without departing from the scope of the embodiment of the present invention.

### EXPLANATION OF REFERENCES

10: learning device
12: communication unit
14: first learning data set database
16: second learning data set database
18: learning model
20: operation unit
22: processor
22A: endoscope image acquisition unit
22B: actual measurement information acquisition unit
22C: imitation image acquisition unit
22D: imitation depth acquisition unit
22E: learning unit
24: RAM
26: ROM
28: display unit
30: bus
109: endoscope system
110: endoscope
111: light source device
112: endoscope processor device
113: display device
120: insertion part
121: hand operation unit
122: universal cord
124: optical range finder
128: imaging element
129: bending operation knob
130: air/water supply button
131: suction button
132: static image-imaging instruction unit
133: treatment tool inlet port
135: light guide
136: signal cable
202: image processing device
204: image acquisition unit
206: processor
208: depth information acquisition device
210: correction unit
212: display controller

What is claimed is:

1. A learning device comprising:
a processor; and
a learning model that estimates depth information of an endoscope image,
wherein the processor is configured to perform
endoscope image acquisition processing of acquiring the endoscope image obtained by imaging a body cavity with an endoscope system,
actual measurement information acquisition processing of acquiring actually measured first depth information corresponding to at least one measurement point in the endoscope image,
imitation image acquisition processing of acquiring an imitation image obtained by imitating an image of the body cavity to be imaged with the endoscope system,
imitation depth acquisition processing of acquiring second depth information including depth information of one or more regions in the imitation image, and
learning processing of causing the learning model to perform learning by using a first learning data set composed of the endoscope image and the first depth information, and a second learning data set composed of the imitation image and the second depth information.

2. The learning device according to claim 1,
wherein the first depth information is acquired by using an optical range finder provided at a distal end of an endoscope of the endoscope system.

3. The learning device according to claim 1,
wherein the imitation image and the second depth information are acquired based on pseudo three-dimensional computer graphics of the body cavity.

4. The learning device according to claim 1,
wherein the imitation image is acquired by imaging a model of the body cavity with the endoscope system, and the second depth information is acquired based on three-dimensional information of the model.

5. The learning device according to claim 1,
wherein the processor is configured to make a first loss weight during the learning processing using the first learning data set and a second loss weight during the learning processing using the second learning data set different from each other.

6. The learning device according to claim 5,
wherein the first loss weight is larger than the second loss weight.

7. A depth information acquisition device comprising:
a trained model in which learning is performed in the learning device according to claim 1.

8. An endoscope system comprising:
the depth information acquisition device according to claim 7;
an endoscope; and
a processor,
wherein the processor is configured to perform
image acquisition processing of acquiring an endoscope image captured with the endoscope,
image input processing of inputting the endoscope image to the depth information acquisition device, and
estimation processing of causing the depth information acquisition device to estimate depth information of the endoscope image.

9. The endoscope system according to claim 8, further comprising:
a correction table corresponding to a second endoscope that differs at least in objective lens from a first endoscope with which the endoscope image of the first learning data set is acquired,
wherein the processor is configured to perform correction processing of correcting the depth information, which is acquired in the estimation processing, by using the correction table in a case where an endoscope image is acquired with the second endoscope.

10. A learning method using a learning device that includes a processor and a learning model that estimates depth information of an endoscope image, the learning method comprising the following steps executed by the processor:
an endoscope image acquisition step of acquiring the endoscope image obtained by imaging a body cavity with an endoscope system;
an actual measurement information acquisition step of acquiring actually measured first depth information corresponding to at least one measurement point in the endoscope image;
an imitation image acquisition step of acquiring an imitation image obtained by imitating an image of the body cavity to be imaged with the endoscope system;
an imitation depth acquisition step of acquiring second depth information including depth information of one or more regions in the imitation image; and
a learning step of causing the learning model to perform learning by using a first learning data set composed of the endoscope image and the first depth information, and a second learning data set composed of the imitation image and the second depth information.

11. A non-transitory, tangible computer-readable recording medium which records thereon a computer instruction for causing, when read by a computer, the computer to execute a learning method for a learning model that estimates depth information of an endoscope image, comprising:
an endoscope image acquisition step of acquiring the endoscope image obtained by imaging a body cavity with an endoscope system;
an actual measurement information acquisition step of acquiring actually measured first depth information corresponding to at least one measurement point in the endoscope image;
an imitation image acquisition step of acquiring an imitation image obtained by imitating an image of the body cavity to be imaged with the endoscope system;
an imitation depth acquisition step of acquiring second depth information including depth information of one or more regions in the imitation image; and
a learning step of causing the learning model to perform learning by using a first learning data set composed of the endoscope image and the first depth information, and a second learning data set composed of the imitation image and the second depth information.
